(19) United States
(12) Patent Application Publication
(10) Pub. No.: US 2021/0338055 A1
(43) Pub. Date: Nov. 4, 2021

(54) FLEXIBLE TUBE INSERTION DEVICE, ENDOSCOPE SYSTEM, AND FLEXIBLE TUBE INSERTION METHOD

(71) Applicant: OLYMPUS CORPORATION, Tokyo (JP)

(72) Inventors: Ryo TEZUKA, Tokyo (JP); Takeshi TAKAHASHI, Tokyo (JP)

(73) Assignee: OLYMPUS CORPORATION, Tokyo (JP)

(21) Appl. No.: 17/373,857

(22) Filed: Jul. 13, 2021

Related U.S. Application Data
(63) Continuation of application No. PCT/JP2019/003191, filed on Jan. 30, 2019.

(51) Int. Cl. A61B 1/005; A61B 34/20

(52) U.S. Cl. CPC A61B 1/0058; A61M 25/0158; A61B 34/20; A61B 1/0055

(57) ABSTRACT

A flexible tube insertion device includes a flexible and elongated insertion portion, a rigidity changeable mechanism extending in a longitudinal direction of the insertion portion in a rigidity changeable range of at least part of the insertion portion and configured to change bending rigidity of the rigidity changeable range, and a processor configured to perform, on the rigidity changeable mechanism, control for changing an insertion shape of the insertion portion being inserted into a subject. The rigidity changeable mechanism performs, in accordance with control by the processor, operation for sequentially increasing bending rigidity of the rigidity changeable range in a direction from a central part of the rigidity changeable range to each end part of the rigidity changeable range.

### CROSS REFERENCE TO RELATED APPLICATION

This application is a continuation application of PCT/JP2019/003191 filed on Jan. 30, 2019, the entire contents of which are incorporated herein by this reference.

### BACKGROUND OF THE INVENTION

### 1. Field of the Invention

The present invention relates to a flexible tube insertion device, an endoscope system, and a flexible tube insertion method.

### 2. Description of the Related Art

In endoscope observation, an insertion operation for inserting an elongated insertion portion having flexibility into a deep part in a subject is performed. For example, in endoscope observation in the medical field, technologies for supporting an insertion operation of an insertion portion being inserted into a subject have been conventionally disclosed.

Specifically, WO 2017/0109987 discloses an endoscope apparatus including an endoscope and an insertion control device, the endoscope being provided with an elongated insertion portion including a flexible tube portion, the insertion control device being configured to control bending rigidity of the flexible tube portion in units of segments divided in advance.

### SUMMARY OF THE INVENTION

A flexible tube insertion device according to an aspect of the present invention includes: an insertion portion having flexibility and an elongated shape; a rigidity changeable mechanism extending in a longitudinal direction of the insertion portion in a rigidity changeable range corresponding to at least a partial range of the insertion portion, the rigidity changeable mechanism being configured to be capable of changing bending rigidity of the rigidity changeable range; and a processor configured to be capable of performing, on the rigidity changeable mechanism, control for changing an insertion shape of the insertion portion being inserted into a subject. The rigidity changeable mechanism is configured to perform, in accordance with control by the processor, operation for sequentially increasing bending rigidity of the rigidity changeable range in a direction from a central part of the rigidity changeable range to each end part of the rigidity changeable range.

An endoscope system according to an aspect of the present invention includes an endoscope and a processor. The endoscope includes an insertion portion having flexibility and an elongated shape, and a rigidity changeable mechanism extending in a longitudinal direction of the insertion portion in a rigidity changeable range corresponding to at least a partial range of the insertion portion, the rigidity changeable mechanism configured to be capable of changing bending rigidity of the rigidity changeable range. The processor is configured to be capable of performing, on the rigidity changeable mechanism, control for changing an insertion shape of the insertion portion being inserted into a subject. The rigidity changeable mechanism is configured to perform, in accordance with control by the processor, operation for sequentially increasing bending rigidity of the rigidity changeable range in a direction from a central part of the rigidity changeable range to each end part of the rigidity changeable range.

A flexible tube insertion method according to an aspect of the present invention is a method of inserting an insertion portion having flexibility and an elongated shape into a subject. The flexible tube insertion method includes: inserting the insertion portion into the subject; and changing, by a rigidity changeable mechanism provided to the insertion portion, an insertion shape of the insertion portion being inserted into the subject by sequentially increasing bending rigidity of a rigidity changeable range corresponding to at least a partial range of the insertion portion in a direction from a central part of the rigidity changeable range to each end part of the rigidity changeable range.

### BRIEF DESCRIPTION OF THE DRAWINGS

FIG. 1 is a diagram illustrating a configuration of a main part of an endoscope system including a flexible tube insertion device according to an embodiment;

FIG. 2 is a block diagram for description of a specific configuration of an endoscope system according to a first embodiment;

FIG. 3 is a diagram for description of configurations and the like of a rigidity changeable mechanism and a rigidity control unit according to the first embodiment;

FIG. 4A is a diagram illustrating an exemplary case in which buckling occurs to an insertion portion inserted inside a subject;

FIG. 4B is a diagram illustrating an exemplary case in which the insertion portion inserted inside the subject passes through a bending site;

FIG. 5 is a diagram for description of configurations and the like of a rigidity changeable mechanism and a rigidity control unit according to a modification of the first embodiment;

FIG. 6A is a diagram illustrating an exemplary case in which buckling occurs to the insertion portion inserted inside the subject;

FIG. 6B is a diagram illustrating an exemplary case in which the insertion portion inserted inside the subject passes through a bending site;

FIG. 7 is a block diagram for description of a specific configuration of an endoscope system according to a second embodiment;

FIG. 8 is a diagram for description of configurations and the like of a rigidity changeable mechanism and a rigidity control unit according to the second embodiment;

FIG. 9 is a diagram for description of operation of the rigidity changeable mechanism according to the second embodiment;

FIG. 10 is a diagram for description of operation of the rigidity changeable mechanism according to the second embodiment;

FIG. 11 is a block diagram for description of configurations and the like of a rigidity changeable mechanism and a rigidity control unit according to a modification of the second embodiment; and

FIG. 12 is a diagram for description of a configuration of the rigidity changeable mechanism according to the modification of the second embodiment.

### DETAILED DESCRIPTION OF THE PREFERRED EMBODIMENTS

Embodiments of the present invention will be described below with reference to the accompanying drawings.

### First Embodiment

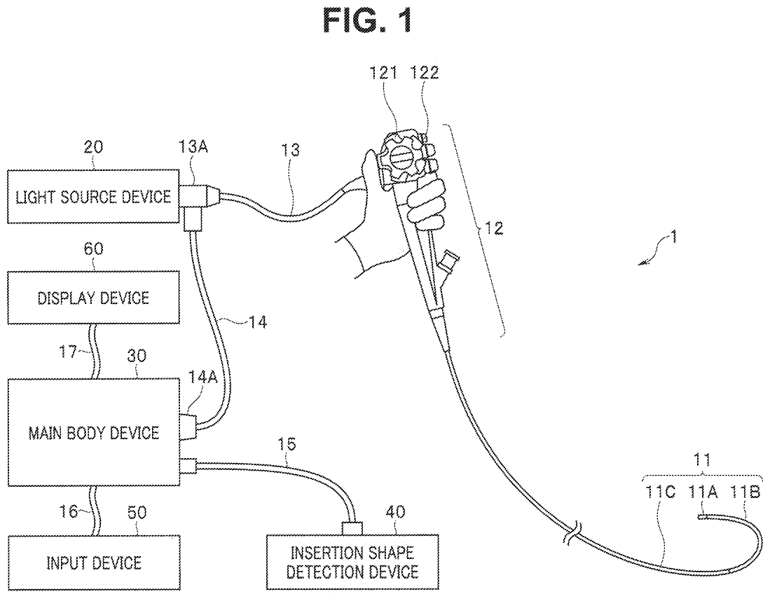

FIGS. 1 to 6B relate to a first embodiment of the present invention.

As illustrated in FIG. 1, for example, an endoscope system 1 includes an endoscope 10, a light source device 20, a main body device 30, an insertion shape detection device 40, an input device 50, and a display device 60.

The endoscope 10 is configured to include an insertion portion 11 to be inserted into a subject, an operation portion 12 provided on a proximal end side of the insertion portion 11, and a universal cord 13 extending from the operation portion 12. The endoscope 10 is removably connected with the light source device 20 through a scope connector 13A provided at an end part of the universal cord 13. The endoscope 10 is also removably connected with the main body device 30 through an electric connector 14A provided at an end part of an electric cable 14 extending from the scope connector 13A. In addition, a light guide (not illustrated) for transmitting illumination light supplied from the light source device 20 is provided inside each of the insertion portion 11, the operation portion 12, and the universal cord 13.

The insertion portion 11 has flexibility and an elongated shape. The insertion portion 11 includes, sequentially from a distal end side, a rigid distal end portion 11A, a bending portion 11B that is bendably formed, and a flexible tube portion 11C that has flexibility and is elongated. In addition, a source coil group 113 (not illustrated in FIG. 1) is provided inside the distal end portion 11A, the bending portion 11B, and the flexible tube portion 11C. In the source coil group, a plurality of source coils, each generate a magnetic field in accordance with a coil drive signal supplied from the main body device 30, are disposed at a predetermined interval in a longitudinal direction of the insertion portion 11.

The distal end portion 11A is provided with an illumination window (not illustrated) through which illumination light transmitted by the light guide provided inside the insertion portion 11 is emitted to an object. The distal end portion 11A is also provided with an image pickup unit 111 (not illustrated in FIG. 1) that is configured to perform operation in accordance with an image pickup control signal supplied from the main body device 30 and is configured to output an image pickup signal by picking up an image of the object illuminated with the illumination light emitted through the illumination window. The image pickup unit 111 includes, for example, an image sensor such as a color CCD.

The bending portion 11B is configure to be able to bend in accordance with an operation of an angle knob 121 provided to the operation portion 12.

A rigidity changeable mechanism 112 (not illustrated in FIG. 1) is configure to be capable of changing a bending rigidity of a rigidity changeable range in accordance with control by the main body device 30 extends in the longitudinal direction of the insertion portion 11 inside the rigidity changeable range corresponding to a predetermined range of the flexible tube portion 11C. Hereinafter, “bending rigidity” is abbreviated as “rigidity” as appropriate for convenience of description. In the present embodiment, the above-described rigidity changeable range may be provided in at least a partial range of the insertion portion 11. A specific configuration and the like of the rigidity changeable mechanism 112 will be described later.

The operation portion 12 has a shape with which the operation portion 12 can be grasped and operated by a user. The operation portion 12 is provided with the angle knob 121 with which an operation for bending the bending portion 11B in four directions, namely, up, down, right, and left directions, intersecting with a longitudinal axis of the insertion portion 11 can be performed. The operation portion 12 is provided with one or more scope switches 122 with which an instruction in accordance with an input operation by the user can be performed.

The light source device 20 includes, as a light source, for example, one or more LEDs or one or more lamps. The light source device 20 is configured to be able to generate illumination light for illuminating inside a subject into which the insertion portion 11 is inserted, and to supply the illumination light to the endoscope 10. The light source device 20 can change a light quantity of the illumination light in accordance with a system control signal supplied from the main body device 30.

The main body device 30 is removably connected with the insertion shape detection device 40 through a cable 15. The main body device 30 is also removably connected with the input device 50 through a cable 16. The main body device 30 is also removably connected with the display device 60 through a cable 17. The main body device 30 performs operation in accordance with instructions from the input device 50 and the scope switches 122. The main body device 30 performs operation for generating an endoscope image based on an image pickup signal outputted from the endoscope 10 and causing the display device 60 to display the generated endoscope image. The main body device 30 generates various kinds of control signals for controlling operation of the endoscope 10 and the light source device 20 and outputs the control signals. The main body device 30 controls a drive state of the rigidity changeable mechanism 112 based on, for example, insertion shape information (to be described later) outputted from the insertion shape detection device 40.

The insertion shape detection device 40 is configured to detect the magnetic field generated by the source coil group 113 provided to the insertion portion 11 and acquires the positions of the plurality of source coils included in the source coil group 113 based on the intensity of the detected magnetic field. The insertion shape detection device 40 calculates an insertion shape of the insertion portion 11 based on the positions of the plurality of source coils acquired as described above, generates insertion shape information indicating the calculated insertion shape, and outputs the insertion shape information to the main body device 30.

The input device 50 includes, for example, one or more input interfaces, such as a mouse, a keyboard, and a touch panel, operated by the user. The input device 50 can output, to the main body device 30, an instruction in accordance with an operation by the user.

The display device 60 includes, for example, a liquid crystal monitor. The display device 60 can display, on a screen, an endoscope image or the like outputted from the main body device 30.

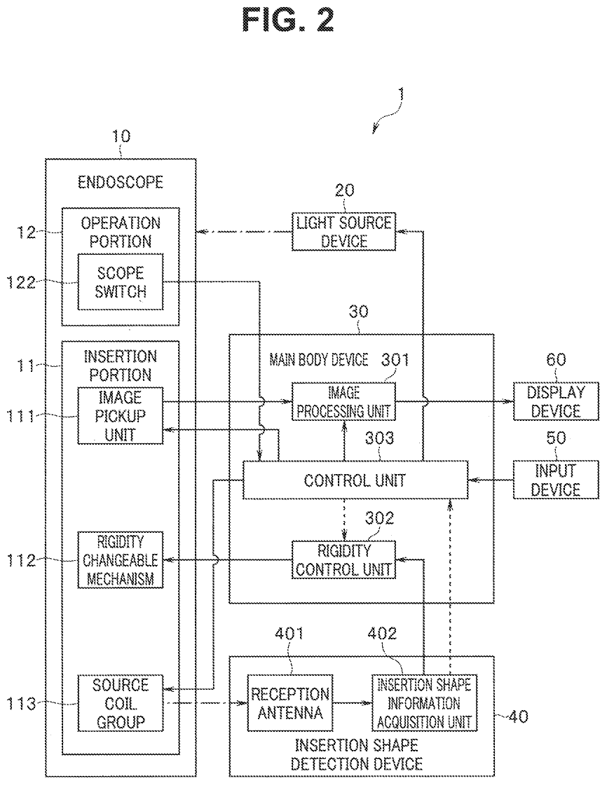

As illustrated in FIG. 2, the main body device 30 includes an image processing unit 301, a rigidity control unit 302, and a control unit 303. FIG. 2 is a block diagram for description of a specific configuration of the endoscope system according to the first embodiment.

In accordance with a system control signal outputted from the control unit 303, the image processing unit 301 is configured to generate an endoscope image by performing predetermined processing on an image pickup signal outputted from the endoscope 10, and output the generated endoscope image to the display device 60.

The rigidity control unit 302 is configured to perform operation for controlling the drive state of the rigidity changeable mechanism 112 based on the insertion shape information outputted from the insertion shape detection device 40. A specific configuration and the like of the rigidity control unit 302 will be described later.

The control unit 303 is configured to generate and output an image pickup control signal for controlling image pickup operation of the image pickup unit 111. The control unit 303 is also configured to generate and output a coil drive signal for driving each source coil included in the source coil group 113. In addition, the control unit 303 is configured to generate a system control signal for performing operation in accordance with instructions from the input device 50 and the scope switches 122, and outputs the generated system control signal to at least one of the light source device 20 or the image processing unit 301.

In the present embodiment, each component of the main body device 30 may be configured as an individual electronic circuit or may be configured as a circuit block in an integrated circuit such as an FPGA (field programmable gate array). In the present embodiment, for example, the main body device 30 may include at least one processor (such as a CPU).

As illustrated in FIG. 2, the insertion shape detection device 40 includes a reception antenna 401 and an insertion shape information acquisition unit 402.

The reception antenna 401 includes, for example, a plurality of coils for three-dimensionally detecting the magnetic field generated by each of the plurality of source coils included in the source coil group 113. The reception antenna 401 is configured to detect the magnetic field generated by each of the plurality of source coils included in the source coil group 113, generates a magnetic field detection signal in accordance with the intensity of the detected magnetic field, and outputs the magnetic field detection signal to the insertion shape information acquisition unit 402.

The insertion shape information acquisition unit 402 is configured to acquire the position of each of the plurality of source coils included in the source coil group 113 based on the magnetic field detection signal outputted from the reception antenna 401. The insertion shape information acquisition unit 402 is configured to calculate the insertion shape of the insertion portion 11 based on the positions of the plurality of source coils acquired as described above, generate insertion shape information indicating the calculated insertion shape, and output the insertion shape information to the rigidity control unit 302.

Specifically, the insertion shape information acquisition unit 402 acquires, as the positions of the plurality of source coils included in the source coil group 113, for example, a plurality of three-dimensional coordinate values in a space coordinate system virtually set with an origin or a reference point at a predetermined position (such as the anus) of the subject into which the insertion portion 11 is inserted. The insertion shape information acquisition unit 402 performs, as processing of calculating the insertion shape of the insertion portion 11, for example, interpolation processing of interpolating the plurality of three-dimensional coordinate values acquired as described above.

In the present embodiment, each component of the insertion shape detection device 40 may be configured as an electronic circuit or may be configured as a circuit block in an integrated circuit such as an FPGA (field programmable gate array). In the present embodiment, for example, the insertion shape detection device 40 may include at least one processor (such as a CPU).

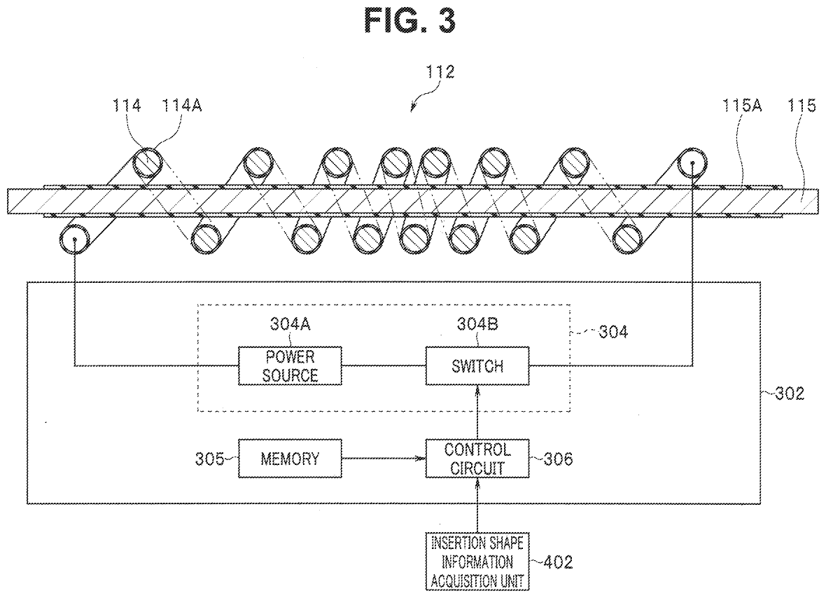

Specific configurations and the like of the rigidity changeable mechanism 112 and the rigidity control unit 302 in the present embodiment will be described below with reference to FIG. 3. FIG. 3 is a diagram for description of configurations and the like of the rigidity changeable mechanism and the rigidity control unit according to the first embodiment.

As illustrated in FIG. 3, the rigidity changeable mechanism 112 is configured as an actuator including a coil heater 114 and a shape-memory member 115.

The coil heater 114 is formed, for example, by cylindrically winding a highly thermally conductive winding wire such as a nichrome wire. The coil heater 114 has such a coil shape that wire winding density gradually decreases in a direction from a central part of one segment corresponding to the entire rigidity changeable range of the insertion portion 11 to each end part of the one segment. A central part of the coil heater 114 is disposed being positioned to a central part of the rigidity changeable mechanism 112, in other words, a central part of the rigidity changeable range of the insertion portion 11. An insulating film 114A is provided on a surface of the coil heater 114. Both ends of the coil heater 114 are electrically connected with a drive circuit 304 (to be described later) of the rigidity control unit 302. The coil heater 114 is configured to generate heat in accordance with control by the rigidity control unit 302.

The shape-memory member 115 is formed, for example, as an elongated member containing shape-memory alloy such as nickel titanium. The shape-memory member 115 is disposed being inserted into an internal space of the coil heater 114. The shape-memory member 115 can change elasticity in accordance with heat generated by the coil heater 114. Specifically, the shape-memory member 115 becomes a high elastic state having restoring force for returning to a straight line shape corresponding to a shape memorized in advance, for example, when heated to a temperature equal to or higher than a temperature TN at least higher than a room temperature by heat generated by the coil heater 114. The shape-memory member 115 becomes a low elastic state not having a restoring force for returning to the straight line shape corresponding to the shape memorized in advance, for example, when not heated to a temperature equal to or higher than the temperature TN due to a factor such as no heat being generated by the coil heater 114. An insulating film 115A is provided to at least a part surrounded by the coil heater 114 on a surface of the shape-memory member 115.

As illustrated in FIG. 3, the rigidity control unit 302 includes the drive circuit 304, a memory 305, and a control circuit 306.

The drive circuit 304 is electrically connected with both ends of the coil heater 114. The drive circuit 304 includes a power source 304A configured to generate a drive current for driving the coil heater 114, and a switch 304B connected in series with the power source 304A and configured to be switched to an “on” state or an “off” state in accordance with control by the control circuit 306.

The memory 305 stores rigidity control information used for control of the switch 304B by the control circuit 306. Specifically, the memory 305 stores the rigidity control information including, for example, information indicating the rigidity changeable range of the insertion portion 11 and information indicating a threshold value corresponding to a predetermined parameter calculated for control of the rigidity changeable mechanism 112.

The control circuit 306 is configured to perform control for setting the switch 304B to the “on” state or the “off” state based on the rigidity control information read from the memory 305 and the insertion shape information outputted from the insertion shape information acquisition unit 402.

Specifically, a flexible tube insertion device according to the present embodiment includes the insertion portion 11, the rigidity changeable mechanism 112, and the rigidity control unit 302.

With the above-described configuration, the rigidity changeable mechanism 112 can perform, in accordance with control by the rigidity control unit 302, operation for sequentially increasing the bending rigidity of the rigidity changeable range of the insertion portion 11 in the direction from the central part of the rigidity changeable range to each end part of the rigidity changeable range.

With the above-described configuration, the rigidity changeable mechanism 112 can change the bending rigidity of the entire rigidity changeable range of the insertion portion 11 as one segment PG, and can perform, in accordance with control by the rigidity control unit 302, operation that increases the bending rigidity at a central part of the one segment PG and then sequentially increases the bending rigidity toward each end part of the one segment PG.

With the above-described configuration, the rigidity control unit 302 is configured to be capable of performing, on the rigidity changeable mechanism 112, control for changing the insertion shape of the insertion portion 11 being inserted into the subject based on the rigidity control information read from the memory 305 and the insertion shape information outputted from the insertion shape information acquisition unit 402.

Subsequently, effects of the present embodiment will be described below.

A user such as an operator connects components of the endoscope system 1 and turns on the endoscope system 1, and then performs, for example, an operation for inserting the insertion portion 11 into the intestinal canal of a subject through the anus. Then, upon such an operation by the user, a magnetic field is generated by each of the plurality of source coils included in the source coil group 113, and a magnetic field detection signal in accordance with the intensity of the magnetic field is outputted from the reception antenna 401.

The insertion shape information acquisition unit 402 acquires the positions of the plurality of source coils included in the source coil group 113 based on the magnetic field detection signal outputted from the reception antenna 401. The insertion shape information acquisition unit 402 calculates the insertion shape of the insertion portion 11 in the intestinal canal based on the positions of the plurality of source coils acquired as described above, generates insertion shape information indicating the calculated insertion shape, and outputs the insertion shape information to the rigidity control unit 302.

The control circuit 306 specifies the rigidity changeable range of the insertion portion 11 based on the rigidity control information read from the memory 305 and the insertion shape information outputted from the insertion shape information acquisition unit 402, calculates a curvature CVA of the specified rigidity changeable range, and determines whether the calculated curvature CVA is equal to or larger than a threshold value TVA. In such a case, the rigidity control information includes the threshold value TVA corresponding to the curvature CVA of the rigidity changeable range of the insertion portion 11.

For example, when having acquired a determination result that the curvature CVA is smaller than the threshold value TVA, the control circuit 306 performs control for setting the switch 304B to the “off” state. Through such control by the control circuit 306, the drive current generated by the power source 304A is not applied to the coil heater 114 and the shape-memory member 115 becomes the low elastic state, and accordingly, the rigidity of the rigidity changeable range of the insertion portion 11 decreases.

When having acquired a determination result that the curvature CVA is equal to or larger than the threshold value TVA, the control circuit 306 performs control for setting the switch 304B to the “on” state. In other words, when having acquired a determination result that the curvature CVA of one segment corresponding to the entire rigidity changeable range of the insertion portion 11 is equal to or larger than the threshold value TVA, the control circuit 306 performs control for generating heat from the coil heater 114. Then, in accordance with such control by the control circuit 306, the drive current generated by the power source 304A is applied to the coil heater 114.

According to the present embodiment, due to the density of the winding wire in the coil heater 114, part of the shape-memory member 115, which is heated by heat generated by the central part of the coil heater 114 transitions from the low elastic state to the high elastic state earlier than part of the shape-memory member 115, which is heated by heat generated by both end parts of the coil heater 114.

Thus, according to the present embodiment, restoring force generated when the shape-memory member 115 transitions from the low elastic state to the high elastic state in accordance with heat generated by the coil heater 114 can be substantially simultaneously generated in two directions, namely, a direction from the central part of the rigidity changeable mechanism 112 to the distal end portion of the insertion portion 11 and a direction from the central part of the rigidity changeable mechanism 112 to the proximal end portion of the insertion portion 11. Thus, according to the present embodiment, since rigidity change along with the above-described restoring force generation substantially simultaneously occurs in the above-described two directions, it is possible to efficiently straighten the insertion portion 11 being buckled inside a subject without providing an excessive load to the subject, and it is possible to increase the rigidity of part of the rigidity changeable range of the insertion portion 11, which is positioned on the distal end side of a bending site inside the subject.

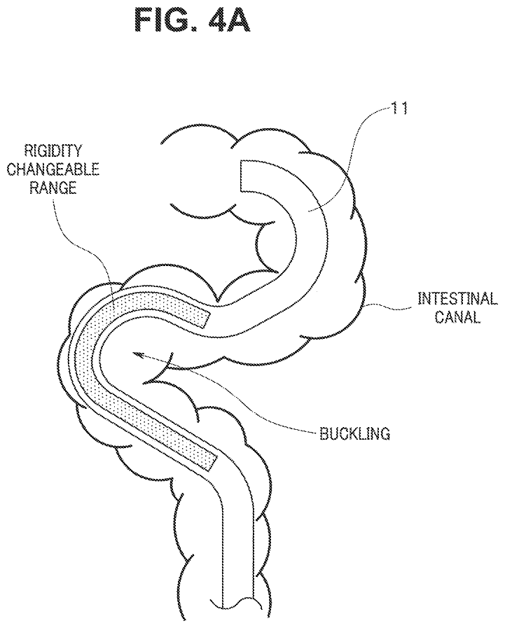

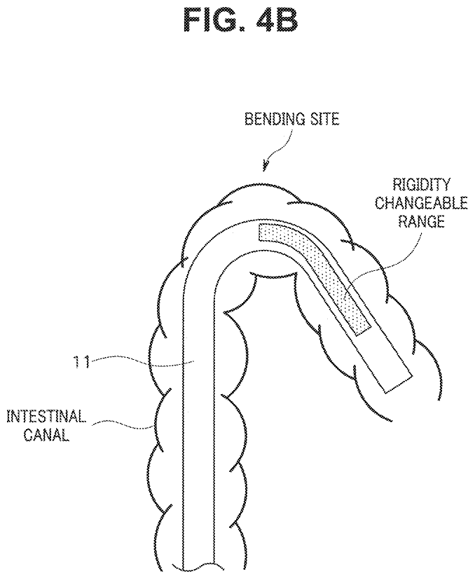

Specifically, according to the present embodiment, it is possible to efficiently straighten the insertion portion 11, for example, when buckling as illustrated in FIG. 4A has occurred to the insertion portion 11 being inserted into the intestinal canal of the subject. In addition, according to the present embodiment, for example, when the insertion portion 11 being inserted into the intestinal canal of the subject passes through a bending site as illustrated in FIG. 4B, it is possible to improve thrust performance of the insertion portion 11 by increasing the rigidity of the rigidity changeable mechanism 112 in a state in which substantially the entire rigidity changeable range of the insertion portion 11 is positioned on the distal end side of the bending site. In such a case, the shape of part of the insertion portion 11, which is positioned on the distal end side of the bending site, deforms closer to a straight line shape along with increase of the rigidity of part of the rigidity changeable mechanism 112, which is positioned on the distal end side of the bending site inside the subject. Thus, according to the present embodiment, it is possible to improve insertion performance when the elongated insertion portion having flexibility is inserted into a deep part in the subject. FIG. 4A is a diagram illustrating an exemplary case in which buckling occurs to the insertion portion being inserted inside the subject. FIG. 4B is a diagram illustrating an exemplary case in which the insertion portion being inserted inside the subject passes through a bending site.

According to the present embodiment, for example, a threshold value TRA corresponding to a curvature radius CRA of the rigidity changeable range of the insertion portion 11 may be included in the rigidity control information. In such a case, control for setting the switch 304B to the “on” state (control for generating heat from the coil heater 114) may be performed when a determination result that the curvature radius CRA is equal to or smaller than the threshold value TRA is obtained, and control for setting the switch 304B to the “off” state may be performed when a determination result that the curvature radius CRA is larger than the threshold value TRA is obtained.

According to the present embodiment, for example, the insertion shape information may be outputted from the insertion shape information acquisition unit 402 to the control unit 303, control for generating an insertion shape image in which the insertion shape of the insertion portion 11 is visualized by using the insertion shape information may be performed by the control unit 303, and processing for causing the display device 60 to display the insertion shape image together with an endoscope image may be performed by the image processing unit 301.

According to the present embodiment, for example, when it is detected by the control unit 303 that an instruction for changing rigidity of the insertion portion 11 (flexible tube portion 11C) is performed through the input device 50 or the scope switches 122, control for setting the switch 304B to the “on” state or the “off” state may be performed by the control circuit 306 in accordance with the instruction.

According to the present embodiment, the insertion shape information indicating the insertion shape of the insertion portion 11 is not limited to acquisition in accordance with a result of detection of a magnetic field generated by each of the plurality of source coils included in the source coil group 113 provided to the insertion portion 11. For example, the insertion shape information may be acquired in accordance with a result of detection of light leakage from the light guide provided to the insertion portion 11 or may be acquired in accordance with a result of detection of ultrasound generated by each of a plurality of ultrasound transducers provided to the insertion portion 11.

According to the present embodiment, for example, when it is detected that the distal end portion of the insertion portion 11 being inserted into a subject has reached a predetermined site in the subject, based on an endoscope image generated by the image processing unit 301, the control circuit 306 may perform control for switching the switch 304B from the “off” state to the “on” state.

According to the present embodiment, for example, the control circuit 306 may perform control for switching the switch 304B from the “off” state to the “on” state when it is detected that the distal end portion of the insertion portion 11 being inserted into the subject has reached a predetermined site in the subject based on the operation state of the angle knob 121 and the amount of insertion of the insertion portion 11 into the subject.

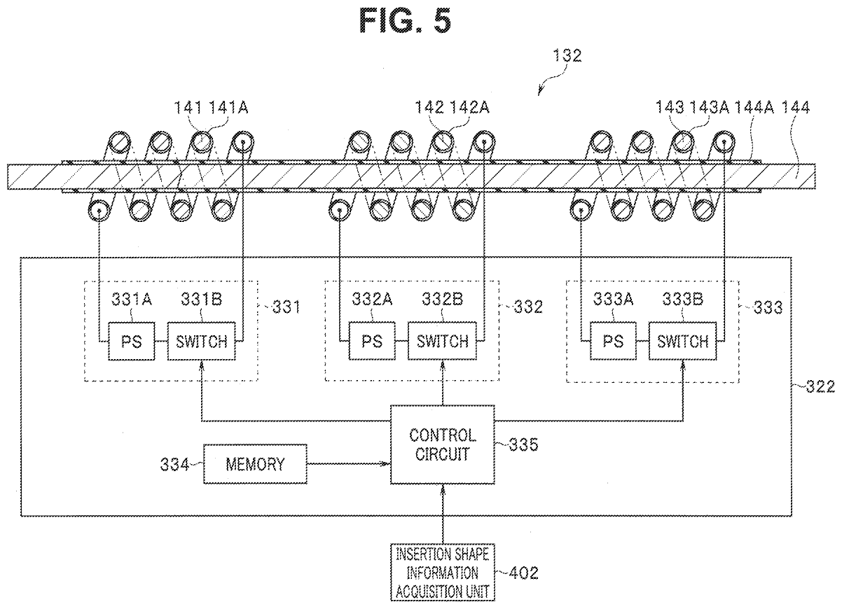

The present embodiment is not limited to a case in which the rigidity changeable mechanism 112 capable of changing rigidity of the rigidity changeable range of the insertion portion 11 as one segment is provided. By applying an appropriate modification, the present embodiment is also applicable to, for example, a case in which a rigidity changeable mechanism capable of changing rigidity for each of a plurality of segments into which the rigidity changeable range is divided is provided. Specifically, the present embodiment is also applicable to, for example, a case in which a rigidity changeable mechanism 132 including three coil heaters 141, 142, and 143 as illustrated in FIG. 5 is provided in a predetermined range (rigidity changeable range) of the flexible tube portion 11C in place of the rigidity changeable mechanism 112. Configurations and the like according to such a modification of the present embodiment will be described below. Hereinafter, specific description related to a part to which an above-described configuration, operation, or the like is applicable is omitted as appropriate for simplification. FIG. 5 is a diagram for description of configurations and the like of a rigidity changeable mechanism and a rigidity control unit according to the modification of the first embodiment.

As illustrated in FIG. 5, the rigidity changeable mechanism 132 is configured as an actuator including the coil heaters 141, 142, and 143 and a shape-memory member 144.

The coil heater 141 is formed, for example, by cylindrically winding a highly thermally conductive winding wire such as a nichrome wire. The coil heater 141 has a coil shape in which wire winding density is substantially constant. For example, the coil heater 141 is disposed being positioned to a position closer to the distal end portion of the insertion portion 11 than the coil heater 142 in the rigidity changeable range of the insertion portion 11. The coil heater 141 is provided near a source coil SA (not illustrated) corresponding to one of the plurality of source coils included in the source coil group 113. An insulating film 141A is provided on a surface of the coil heater 141. Both ends of the coil heater 141 are electrically connected with a drive circuit 331 (to be described later) of a rigidity control unit 322. The coil heater 141 is configured to generate heat in accordance with control by the rigidity control unit 322.

The coil heater 142 is formed, for example, by cylindrically winding a highly thermally conductive winding wire such as a nichrome wire. The coil heater 142 has a coil shape in which wire winding density is substantially constant. The coil heater 142 is disposed between the coil heater 141 and the coil heater 143. The coil heater 142 is disposed at the central part of the rigidity changeable range of the insertion portion 11. Specifically, a central part of the coil heater 142 is disposed being positioned to a central part of the rigidity changeable mechanism 132. The coil heater 142 is provided near a source coil SB (not illustrated) corresponding to one source coil disposed at a position closer to the proximal end side of the insertion portion 11 than the source coil SA among the plurality of source coils included in the source coil group 113. An insulating film 142A is provided on a surface of the coil heater 142. Both ends of the coil heater 142 are electrically connected with a drive circuit 332 (to be described later) of the rigidity control unit 322. The coil heater 142 is configured to generate heat in accordance with control by the rigidity control unit 322.

The coil heater 143 is formed, for example, by cylindrically winding a highly thermally conductive winding wire such as a nichrome wire. The coil heater 143 has a coil shape in which wire winding density is substantially constant. For example, the coil heater 143 is disposed being positioned to a position closer to the proximal end portion of the insertion portion 11 than the coil heater 142 in the rigidity changeable range of the insertion portion 11. The coil heater 143 is provided near a source coil SC (not illustrated) corresponding to one source coil disposed at a position closer to the proximal end side of the insertion portion 11 than the source coil SB among the plurality of source coils included in the source coil group 113. An insulating film 143A is provided on a surface of the coil heater 143. Both ends of the coil heater 143 are electrically connected with a drive circuit 333 (to be described later) of the rigidity control unit 322. The coil heater 143 is configured to generate heat in accordance with control by the rigidity control unit 322.

The shape-memory member 144 is formed, for example, as an elongated member containing shape-memory alloy such as nickel titanium. The shape-memory member 144 is disposed being inserted into internal spaces of the coil heaters 141, 142, and 143. The shape-memory member 144 is configured to be able to change elasticity in accordance with heat generated by at least one of the coil heater 141, 142, or 143. Specifically, in accordance with heat generated by at least one of the coil heater 141, 142, or 143, part of the shape-memory member 144, which is heated to a temperature equal to or higher than a temperature TN becomes the high elastic state, and part of the shape-memory member 144, which is not heated to a temperature equal to or higher than the temperature TN becomes the low elastic state. An insulating film 144A is provided to at least a part surrounded by the coil heaters 141, 142, and 143 on a surface of the shape-memory member 144.

Specifically, with the configuration of the rigidity changeable mechanism 132 as described above, the entire rigidity changeable range of the insertion portion 11 is divided into three segments, namely, a segment PA (not illustrated) corresponding to the position of the source coil SA, a segment PB (not illustrated) corresponding to the position of the source coil SB, and a segment PC (not illustrated) corresponding to the position of the source coil SC. With the configuration of the rigidity changeable mechanism 132 as described above, the coil heater 141 is disposed at a position corresponding to the segment PA, the coil heater 142 is disposed at a position corresponding to the segment PB, and the coil heater 143 is disposed at a position corresponding to the segment PC. With the configuration as described above, the rigidity changeable mechanism 132 can change rigidity for each of the three segments PA, PB, and PC.

The rigidity control unit 322 is provided to the main body device 30 in place of the rigidity control unit 302. The rigidity control unit 322 is configured to perform operation for controlling a drive state of the rigidity changeable mechanism 132 based on the insertion shape information outputted from the insertion shape information acquisition unit 402. As illustrated in FIG. 5, the rigidity control unit 322 includes the drive circuits 331, 332, and 333, a memory 334, and a control circuit 335.

The drive circuit 331 is electrically connected with both ends of the coil heater 141. The drive circuit 331 includes a power source (abbreviated as PS in FIG. 5) 331A configured to generate a drive current for driving the coil heater 141, and a switch 331B connected in series with the power source 331A and configured to be switched the “on” state or the “off” state in accordance with control by the control circuit 335.

The drive circuit 332 is electrically connected with both ends of the coil heater 142. The drive circuit 332 includes a power source (abbreviated as PS in FIG. 5) 332A configured to generate a drive current for driving the coil heater 142, and a switch 332B connected in series with the power source 332A and configured to be switched the “on” state or the “off” state in accordance with control by the control circuit 335.

The drive circuit 333 is electrically connected with both ends of the coil heater 143. The drive circuit 333 includes a power source (abbreviated as PS in FIG. 5) 333A configured to generate a drive current for driving the coil heater 143, and a switch 333B connected in series with the power source 333A and configured to be switched the “on” state or the “off” state in accordance with control by the control circuit 335.

The memory 334 stores rigidity control information used for control of the switches 331B, 332B, and 333B by the control circuit 335. Specifically, the memory 334 stores the rigidity control information including, for example, information indicating the rigidity changeable range of the insertion portion 11, information that can specify the three segments PA, PB, and PC included in the rigidity changeable range, and information indicating a threshold value corresponding to a predetermined parameter calculated for control of the rigidity changeable mechanism 132.

The control circuit 335 is configured to perform control for individually setting the switches 331B, 332B, and 333B to the “on” state or the “off” state based on the rigidity control information read from the memory 334 and the insertion shape information outputted from the insertion shape information acquisition unit 402.

Specifically, a flexible tube insertion device according to the present modification includes the insertion portion 11, the rigidity changeable mechanism 132, and the rigidity control unit 322.

With the above-described configuration, the rigidity changeable mechanism 132 can perform, in accordance with control by the rigidity control unit 322, operation for sequentially increasing the bending rigidity of the rigidity changeable range of the insertion portion 11 in the direction from the central part of the rigidity changeable range to each end part of the rigidity changeable range.

With the above-described configuration, the rigidity changeable mechanism 132 can change bending rigidity for each of a plurality of segments into which the entire rigidity changeable range of the insertion portion 11 is divided.

With the above-described configuration, the rigidity control unit 322 can perform, on the rigidity changeable mechanism 132, control for changing the insertion shape of the insertion portion 11 being inserted into a subject based on the rigidity control information read from the memory 334 and the insertion shape information outputted from the insertion shape information acquisition unit 402.

Subsequently, effects of the present modification will be described below.

The control circuit 335 specifies each of the segments PA, PB, and PC included in the rigidity changeable range of the insertion portion 11 based on the rigidity control information read from the memory 334 and the insertion shape information outputted from the insertion shape information acquisition unit 402. The control circuit 335 calculates a curvature CVB of the segment PB specified as described above and determines whether the calculated curvature CVB is equal to or larger than a threshold value TVB. In such a case, the rigidity control information includes the threshold value TVB corresponding to the curvature CVB of the segment PB positioned at the central part of the rigidity changeable range of the insertion portion 11.

For example, when having acquired a determination result that the curvature CVB is smaller than the threshold value TVB, the control circuit 335 performs control for setting the switches 331B, 332B, and 333B to the “off” state. Through such control by the control circuit 335, the drive current generated by the power sources 331A, 332A, and 333A is not applied to the coil heaters 141, 142, and 143 and the shape-memory member 144 becomes the low elastic state, and accordingly, the rigidity of the rigidity changeable range of the insertion portion 11 decreases.

When having acquired a determination result that the curvature CVB is equal to or larger than the threshold value TVB, the control circuit 335 performs control for setting the switch 332B to the “on” state. After a certain time has elapsed since the control for setting the switch 332B to the “on” state is performed, the control circuit 335 performs control for simultaneously setting the switches 331B and 333B to the “on” state. Through such control by the control circuit 335, drive current application from the power source 332A to the coil heater 142 is started at a timing TA when the determination result that the curvature CVB is equal to or larger than the threshold value TVB is obtained. Through control by the control circuit 335 as described above, drive current application from the power source 331A to the coil heater 141 and drive current application from the power source 333A to the coil heater 143 are started at a timing TB a certain time after the timing TA.

Specifically, the control circuit 335 performs control that operates the rigidity changeable mechanism to increase the bending rigidity of the segment PB belonging to the central part of the rigidity changeable range of the insertion portion 11 and then sequentially increase the bending rigidity toward the segments PA and PC belonging to both end parts of the rigidity changeable range.

According to the present modification, since drive current application to the coil heaters 141 and 143 is started after drive current application to the coil heater 142 is started, the segments PA and PC of the shape-memory member 144 substantially simultaneously transition to the high elastic state after the segment PB of the shape-memory member 144 transitions to the high elastic state.

Thus, according to the present modification, restoring force generated when the shape-memory member 144 transitions from the low elastic state to the high elastic state in accordance with heat generated by the coil heaters 141, 142, and 143 can be substantially simultaneously generated in two directions, namely, a direction from the central part of the rigidity changeable mechanism 132 to the distal end portion of the insertion portion 11 and a direction from the central part of the rigidity changeable mechanism 132 to the proximal end portion of the insertion portion 11. Thus, according to the present modification, since rigidity change along with the above-described restoring force generation substantially simultaneously occurs in the above-described two directions, it is possible to efficiently straighten the insertion portion 11 being buckled inside a subject without providing an excessive load to the subject, and it is possible to increase the rigidity of part of the rigidity changeable range of the insertion portion 11, which is positioned on the distal end side of a bending site inside the subject.

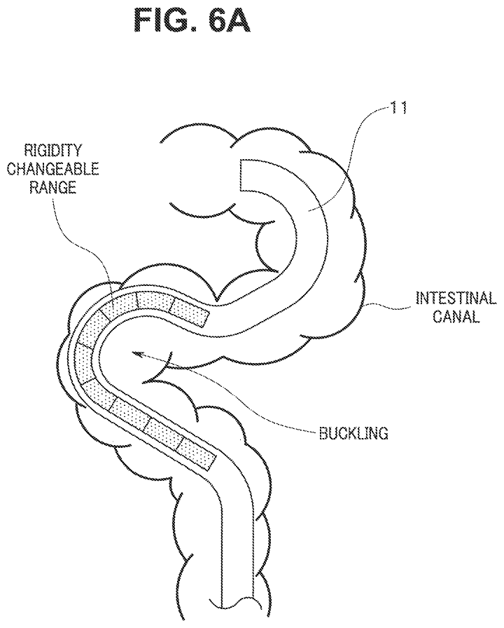

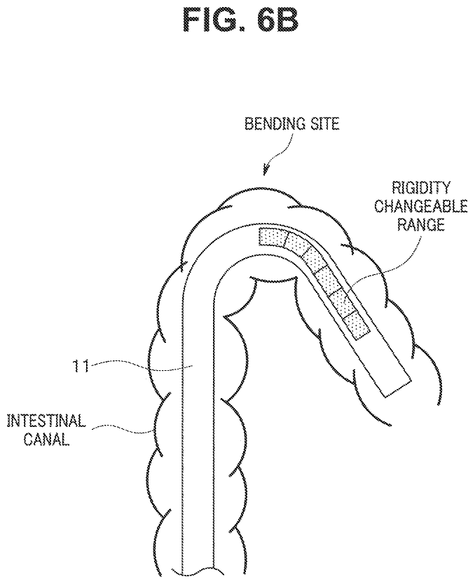

Specifically, according to the present modification, it is possible to efficiently straighten the insertion portion 11, for example, when buckling as illustrated in FIG. 6A has occurred to the insertion portion 11 being inserted into the intestinal canal of the subject. According to the present modification, for example, when the insertion portion 11 being inserted into the intestinal canal of the subject passes through a bending site as illustrated in FIG. 6B, it is possible to improve thrust performance of the insertion portion 11 by increasing the rigidity of the rigidity changeable mechanism 132 in a state in which substantially the entire rigidity changeable range of the insertion portion 11 is positioned on the distal end side of the bending site. In such a case, the shape of part of the insertion portion 11, which is positioned on the distal end side of the bending site deforms closer to a straight line shape along with increase of the rigidity of part of the rigidity changeable mechanism 132, which is positioned on the distal end side of the bending site inside the subject. Thus, according to the present modification, it is possible to improve insertion performance when the elongated insertion portion having flexibility is inserted into a deep part in the subject. FIG. 6A is a diagram illustrating an exemplary case in which buckling occurs to the insertion portion being inserted inside the subject. FIG. 6B is a diagram illustrating an exemplary case in which the insertion portion being inserted inside the subject passes through a bending site.

According to the present modification, for example, the rigidity control information may include a threshold value TRB corresponding to a curvature radius CRB of the segment PB positioned at the central part of the rigidity changeable range of the insertion portion 11. In such a case, control for setting the three switches 331B, 332B, and 333B to the “on” state in the stated order may be performed when a determination result that the curvature radius CRB is equal to or smaller than the threshold value TRB is obtained, and control for setting the three switches to the “off” state in an order opposite to the above-described order may be performed when a determination result that the curvature radius CRB is larger than the threshold value TRB is obtained.

According to the present modification, for example, when the rigidity changeable range of the insertion portion 11 is divided into four or more segments, control may be performed by considering that a plurality of segments belong to the central part of the rigidity changeable range.

Specifically, according to the present modification, the rigidity control unit 322 may perform control that operates the rigidity changeable mechanism 132 to increase the bending rigidity of one or more segments belonging to the central part of the rigidity changeable range of the insertion portion 11 among a plurality of segments included in the rigidity changeable range and then sequentially increase the bending rigidity toward segments belonging to both end parts of the rigidity changeable range among the plurality of segments. According to the present modification, when the curvature of a predetermined segment included in one or more segments belonging to the central part of the rigidity changeable range of the insertion portion 11 is equal to or larger than a threshold value or when the curvature radius of the predetermined segment is equal to or smaller than a threshold value, the rigidity control unit 322 may perform predetermined control for generating heat from one or more coil heaters disposed at the central part of the rigidity changeable range and then may perform the predetermined control on each coil heater other than the one or more coil heaters in ascending order of distance to the central part of the rigidity changeable range.

### Second Embodiment

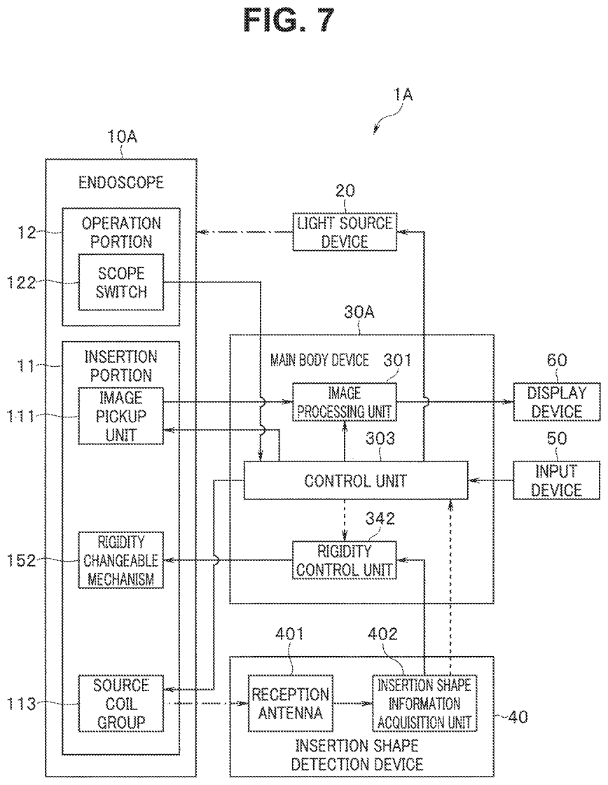

FIGS. 7 to 12 relate to a second embodiment of the present invention.

In the present embodiment, detailed description of a part having a configuration or the like same as a configuration or the like in the first embodiment is omitted as appropriate, and description is mainly performed on a part having a configuration or the like different from a configuration or the like in the first embodiment.

As illustrated in FIG. 7, an endoscope system 1A includes an endoscope 10A, the light source device 20, a main body device 30A, the insertion shape detection device 40, the input device 50, and the display device 60. FIG. 7 is a block diagram for description of a specific configuration of the endoscope system according to the second embodiment.

The endoscope 10A has a configuration in which a rigidity changeable mechanism 152 is provided in place of the rigidity changeable mechanism 112 in the endoscope 10. Specifically, the rigidity changeable mechanism 152 is provided inside the rigidity changeable range of the insertion portion 11 (flexible tube portion 11C) of the endoscope 10A.

The main body device 30A has a configuration in which a rigidity control unit 342 is provided in place of the rigidity control unit 302 in the main body device 30.

The rigidity control unit 342 is configured to perform operation for controlling the rigidity changeable mechanism 152 based on the insertion shape information outputted from the insertion shape detection device 40.

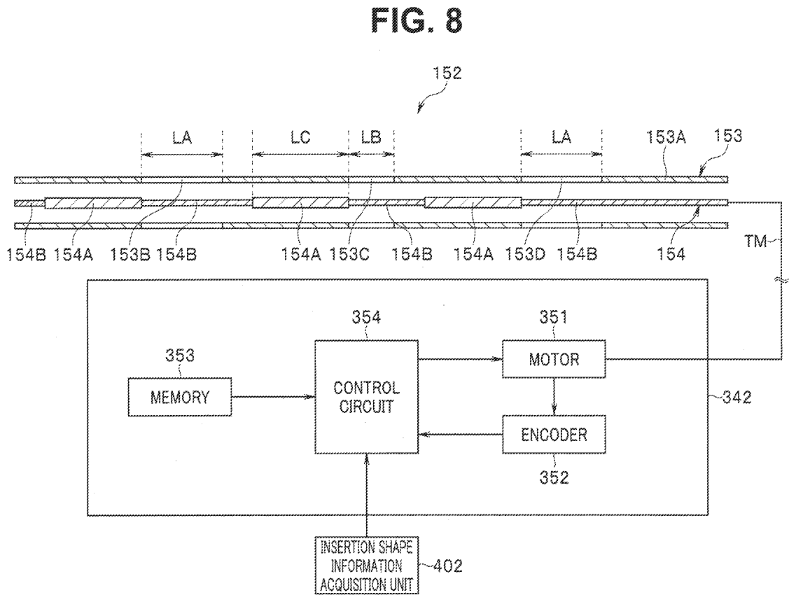

Specific configurations and the like of the rigidity changeable mechanism 152 and the rigidity control unit 342 in the present embodiment will be described below with reference to FIG. 8. FIG. 8 is a diagram for description of configurations and the like of a rigidity changeable mechanism and a rigidity control unit according to the second embodiment.

The rigidity changeable mechanism 152 extends in the longitudinal direction of the insertion portion 11 inside the rigidity changeable range in the flexible tube portion 11C. The rigidity changeable mechanism 152 can change the bending rigidity of the rigidity changeable range of the insertion portion 11 in accordance with control by the main body device 30A. As illustrated in FIG. 8, the rigidity changeable mechanism 152 includes a sheath member 153 and a bar member 154. In other words, the rigidity changeable mechanism 152 has one rigidity changeable structure including the sheath member 153 and the bar member 154.

The sheath member 153 has, for example, an elongated cylindrical shape. The sheath member 153 is disposed being fixed inside the insertion portion 11 (flexible tube portion 11C). For example, three slits 153B, 153C, and 153D are formed at an outer cover 153A of the sheath member 153.

The slit 153B is formed, for example, at a position closer to the distal end portion of the insertion portion 11 than the slit 153C in the rigidity changeable range of the insertion portion 11. The slit 153B is formed, for example, by cutting the outer cover 153A in a longitudinal direction of the sheath member 153 by a length LA.

The slit 153C is formed at the central part of the rigidity changeable range of the insertion portion 11, in other words, the central part of the rigidity changeable mechanism 132. The slit 153C is formed, for example, by cutting the outer cover 153A in the longitudinal direction of the sheath member 153 by a length LB (<LA).

The slit 153D is formed, for example, at a position closer to the proximal end portion of the insertion portion 11 than the slit 153C in the rigidity changeable range of the insertion portion 11. The slit 153D is formed, for example, by cutting the outer cover 153A in the longitudinal direction of the sheath member 153 by the length LA.

In other words, a plurality of slits having lengths that gradually increase in a direction from a central part of one segment corresponding to the entire rigidity changeable range of the insertion portion 11 to each end part of the one segment are formed at the outer cover 153A of the sheath member 153.

The bar member 154 is disposed being inserted into an internal space of the sheath member 153. The bar member 154 has, for example, an elongated cylinder shape. The bar member 154 is disposed being slidable inside the sheath member 153. An end part of the bar member 154 on the proximal end side is connected with the rigidity control unit 342 through a pulling member TM such as a wire. The bar member 154 together with the pulling member TM can move forward and backward inside the sheath member 153. In addition, a large diameter portion 154A (large diameter site) having a length LC equal to or larger than the length LA and having a relatively large diameter and a small diameter portion 154B (small diameter site) having a relatively small diameter are alternately provided in a longitudinal direction of the bar member 154. The length LC of each large diameter portion 154A in the bar member 154 may be equal to or longer than a maximum length among the lengths of the plurality of slits formed at the outer cover 153A of the sheath member 153.

As illustrated in FIG. 8, the rigidity control unit 342 includes a motor 351, an encoder 352, a memory 353, and a control circuit 354.

The motor 351 is connected with the end part of the bar member 154 on the proximal end side through the pulling member TM. The motor 351 can change a pulling amount of the pulling member TM (length by which the pulling member TM is wound) by rotating in accordance with control by the control circuit 354.

For example, the encoder 352 is configured to detect a current rotational amount and a current rotational direction of the motor 351 as a rotation state of the motor 351 and outputs, to the control circuit 354, rotation state information indicating the detected rotation state of the motor 351.

The memory 353 stores rigidity control information used for control of the motor 351 by the control circuit 354. Specifically, the memory 353 stores the rigidity control information including, for example, information indicating the rigidity changeable range of the insertion portion 11 and information indicating a threshold value corresponding to a predetermined parameter calculated for control of the rigidity changeable mechanism 152.

The control circuit 354 is configured to control the rotational amount and rotational direction of the motor 351 based on the rotation state information outputted from the encoder 352, the rigidity control information read from the memory 353, and the insertion shape information outputted from the insertion shape information acquisition unit 402.

Specifically, a flexible tube insertion device according to the present embodiment includes the insertion portion 11, the rigidity changeable mechanism 152, and the rigidity control unit 342.

With the above-described configuration, the rigidity changeable mechanism 152 can perform, in accordance with control by the rigidity control unit 342, operation for sequentially increasing the bending rigidity of the rigidity changeable range of the insertion portion 11 in the direction from the central part of the rigidity changeable range to each end part of the rigidity changeable range.

With the above-described configuration, the rigidity changeable mechanism 152 can change the bending rigidity of the entire rigidity changeable range of the insertion portion 11 as one segment PH and can perform, in accordance with control by the rigidity control unit 342, operation to increase the bending rigidity of a central part of the one segment PH and then sequentially increase the bending rigidity toward each end part of the one segment PH.

With the above-described configuration, the bar member 154 of the rigidity changeable mechanism 152 can change the position of the bar member 154 relative to the sheath member 153 in accordance with control by the rigidity control unit 342.

With the above-described configuration, the rigidity control unit 342 can perform, on the rigidity changeable mechanism 152, control for changing the insertion shape of the insertion portion 11 being inserted into a subject based on the rigidity control information read from the memory 353 and the insertion shape information outputted from the insertion shape information acquisition unit 402.

Subsequently, effects of the present embodiment will be described below.

A user such as an operator connects components of the endoscope system 1A and turns on the endoscope system 1A and then performs, for example, an operation for inserting the insertion portion 11 into the intestinal canal of a subject through the anus. Then, upon such an operation by the user, a magnetic field is generated by each of the plurality of source coils included in the source coil group 113, and a magnetic field detection signal in accordance with the intensity of the magnetic field is outputted from the reception antenna 401.

The insertion shape information acquisition unit 402 acquires the positions of the plurality of source coils included in the source coil group 113 based on the magnetic field detection signal outputted from the reception antenna 401. The insertion shape information acquisition unit 402 calculates the insertion shape of the insertion portion 11 in the intestinal canal based on the positions of the plurality of source coils acquired as described above, generates insertion shape information indicating the calculated insertion shape, and outputs the insertion shape information to the rigidity control unit 342.

The control circuit 354 specifies the rigidity changeable range of the insertion portion 11 based on the rigidity control information read from the memory 353 and the insertion shape information outputted from the insertion shape information acquisition unit 402, calculates a curvature CVC of the specified rigidity changeable range, and determines whether the calculated curvature CVC is equal to or larger than a threshold value TVC. In such a case, the rigidity control information includes the threshold value TVC corresponding to the curvature CVC of the rigidity changeable range of the insertion portion 11.

For example, when having acquired a determination result that the curvature CVC is smaller than the threshold value TVC, the control circuit 354 performs, based on the rotation state information outputted from the encoder 352, control for rotating the motor 351 so that the pulling amount of the pulling member TM becomes equal to a pulling amount PK. Then, the bar member 154 is pulled in accordance with such control by the control circuit 354, and a relative positional relation between the sheath member 153 and the bar member 154 changes, and accordingly, for example, the rigidity changeable mechanism 152 becomes a state as illustrated in FIG. 8.

In the rigidity changeable mechanism 152 in the state as illustrated in FIG. 8, each large diameter portion 154A is disposed at a position where the large diameter portion 154A is surrounded by the outer cover 153A, and each small diameter portion 154B is disposed at a position where the small diameter portion 154B blocks the corresponding one of the three slits 153B, 153C, and 153D. Thus, as the rigidity changeable mechanism 152 becomes the state as illustrated in FIG. 8, the rigidity of the entire rigidity changeable range of the insertion portion 11 uniformly decreases.

For example, when having acquired a determination result that the curvature CVC is equal to or larger than the threshold value TVC, the control circuit 354 performs, based on the rotation state information outputted from the encoder 352, control for rotating the motor 351 so that the pulling amount of the pulling member TM becomes equal to a pulling amount PM (>PK). In other words, when having acquired a determination result that the curvature CVC of one segment corresponding to the entire rigidity changeable range of the insertion portion 11 is equal to or larger than the threshold value TVC, the control circuit 354 performs control for displacing the bar member 154 so that the position of each large diameter portion 154A coincides with the position of the corresponding one of the slits 153B, 153C, and 153D.

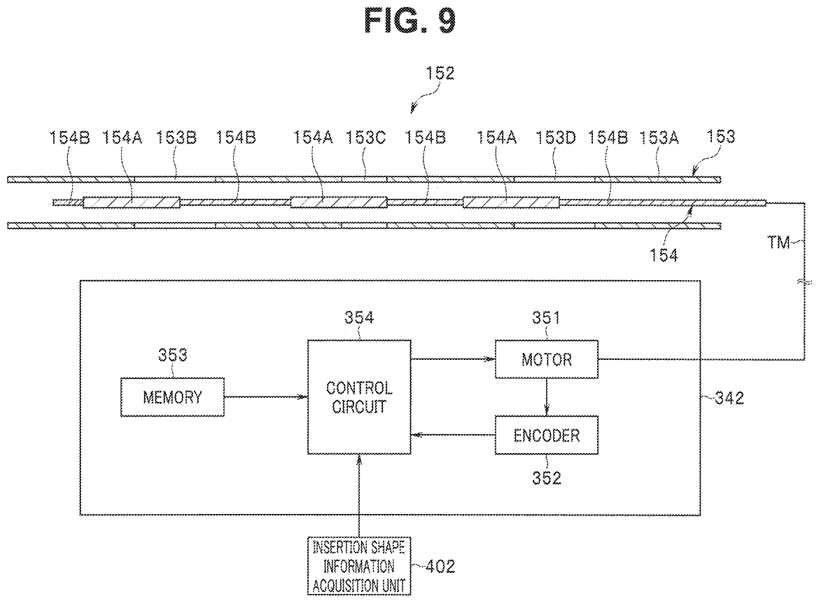

When the pulling amount of the pulling member TM has reached a pulling amount PL (PK<PL<PM), for example, the rigidity changeable mechanism 152 changes from the state as illustrated in FIG. 8 to a state as illustrated in FIG. 9. FIG. 9 is a diagram for description of operation of the rigidity changeable mechanism according to the second embodiment.

In the rigidity changeable mechanism 152 in the state as illustrated in FIG. 9, a large diameter portion 154A is disposed at a position where the large diameter portion 154A blocks the slit 153C, and each pair of a large diameter portion 154A and a small diameter portion 154B are disposed at positions where the large diameter portion 154A and the small diameter portion 154B block the corresponding one of the slits 153B and 153D. Thus, as the rigidity changeable mechanism 152 becomes the state as illustrated in FIG. 9, the rigidity of the central part of the rigidity changeable range of the insertion portion 11 becomes highest and the rigidity of the rigidity changeable range gradually decreases from the central part toward each end part.

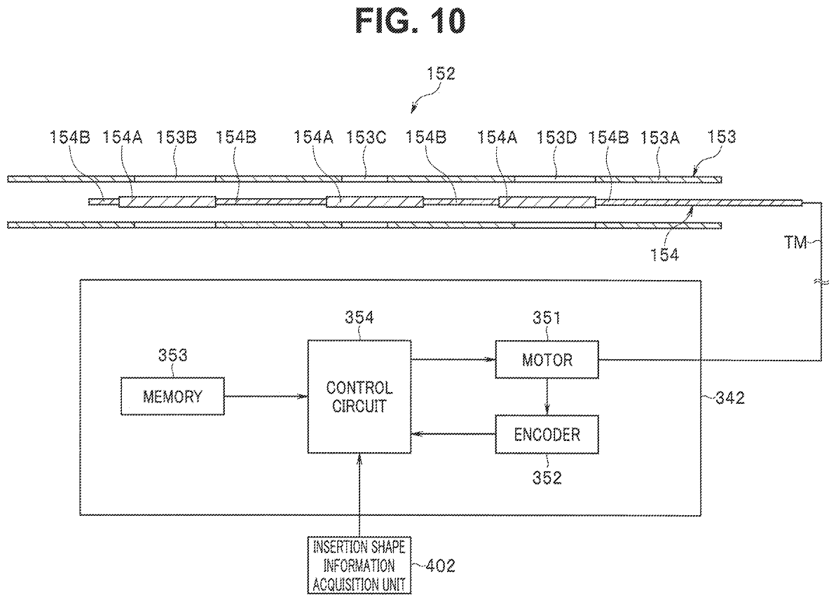

When the pulling amount of the pulling member TM has reached the pulling amount PM, for example, the rigidity changeable mechanism 152 changes from the state as illustrated in FIG. 9 to a state as illustrated in FIG. 10. FIG. 10 is a diagram for description of operation of the rigidity changeable mechanism according to the second embodiment.

In the rigidity changeable mechanism 152 in the state as illustrated in FIG. 10, each small diameter portion 154B is disposed at a position where the small diameter portion 154B is surrounded by the outer cover 153A, and each large diameter portion 154A is disposed at a position where the large diameter portion 154A blocks the corresponding one of the three slits 153B, 153C, and 153D. Thus, as the rigidity changeable mechanism 152 becomes the state as illustrated in FIG. 10, the rigidity of the entire rigidity changeable range of the insertion portion 11 uniformly increases.

As described above, according to the present embodiment, force for deforming the insertion portion 11 closer to a straight line shape can be generated by changing the relative positional relation between the sheath member 153 and the bar member 154. According to the present embodiment, force for deforming the insertion portion 11 closer to a straight line shape can be substantially simultaneously generated in two directions, namely, a direction from a central part of the rigidity changeable mechanism 152 to the distal end portion of the insertion portion 11 and a direction from the central part of the rigidity changeable mechanism 152 to the proximal end portion of the insertion portion 11. Thus, according to the present embodiment, since rigidity change along with the above-described force generation substantially simultaneously occurs in the above-described two directions, it is possible to efficiently straighten the insertion portion 11 being buckled inside a subject without providing an excessive load to the subject, and it is possible to increase the rigidity of part of the rigidity changeable range of the insertion portion 11, which is positioned on the distal end side of a bending site inside the subject.

Specifically, according to the present embodiment, it is possible to efficiently straighten the insertion portion 11, for example, when buckling as illustrated in FIG. 4A has occurred to the insertion portion 11 being inserted into the intestinal canal of the subject. According to the present embodiment, for example, when the insertion portion 11 being inserted into the intestinal canal of the subject passes through a bending site as illustrated in FIG. 4B, it is possible to improve thrust performance of the insertion portion 11 by increasing the rigidity of the rigidity changeable mechanism 152 in a state in which substantially the entire rigidity changeable range of the insertion portion 11 is positioned on the distal end side of the bending site. In such a case, the shape of part of the insertion portion 11, which is positioned on the distal end side of the bending site deforms closer to a straight line shape along with increase of the rigidity of part of the rigidity changeable mechanism 152, which is positioned on the distal end side of the bending site inside the subject. Thus, according to the present embodiment, it is possible to improve insertion performance when the elongated insertion portion having flexibility is inserted into a deep part in the subject.

According to the present embodiment, for example, the rigidity control information may include a threshold value TRC corresponding to a curvature radius CRC of the rigidity changeable range of the insertion portion 11. In such a case, control for rotating the motor 351 so that the pulling amount of the pulling member TM becomes equal to the pulling amount PM may be performed when a determination result that the curvature radius CRC is equal to or smaller than the threshold value TRC is obtained, and control for rotating the motor 351 so that the pulling amount of the pulling member TM becomes equal to the pulling amount PK may be performed when a determination result that the curvature radius CRC is larger than the threshold value TRC is obtained.

According to the present embodiment, for example, when it is detected by the control unit 303 that an instruction for changing the rigidity of the insertion portion 11 (flexible tube portion 11C) is performed through the input device 50 or the scope switches 122, the control circuit 354 may perform control for changing the pulling amount of the pulling member TM by the motor 351 in accordance with the instruction.

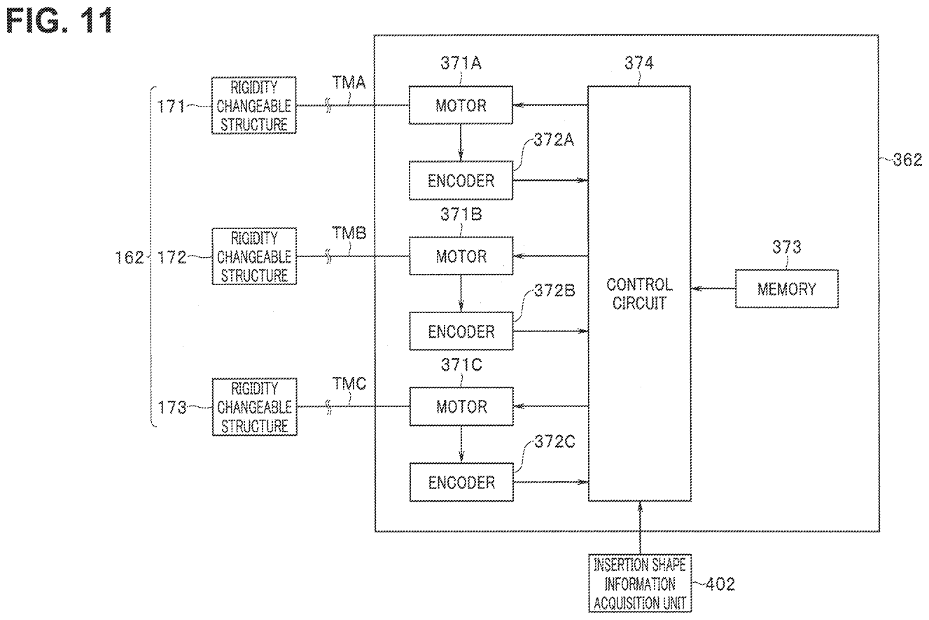

The present embodiment is not limited to a case in which the rigidity changeable mechanism 152 capable of changing rigidity of the rigidity changeable range of the insertion portion 11 as one segment is provided. By applying an appropriate modification, the present embodiment is also applicable to, for example, a case in which a rigidity changeable mechanism capable of changing rigidity for each of a plurality of segments into which the rigidity changeable range is divided is provided. Specifically, the present embodiment is also applicable to, for example, a case in which a rigidity changeable mechanism 162 including three rigidity changeable structures 171, 172, and 173 as illustrated in FIG. 11 is provided in a predetermined range of the flexible tube portion 11C in place of the rigidity changeable mechanism 152. Configurations and the like according to such a modification of the present embodiment will be described below. The rigidity changeable structures 171, 172, and 173 according to the present modification have configurations substantially same as one another and are controlled by control methods substantially same as one another. Thus, the following description will be mainly made on specific configurations and the like of the rigidity changeable structure 172, but description of specific configurations and the like of the rigidity changeable structures 171 and 173 will be omitted as appropriate. FIG. 11 is a block diagram for description of configurations and the like of a rigidity changeable mechanism and a rigidity control unit according to a modification of the second embodiment.

As illustrated in FIG. 11, the rigidity changeable mechanism 162 includes the rigidity changeable structures 171, 172, and 173.

For example, the rigidity changeable structure 171 is disposed being positioned to a position closer to the distal end portion of the insertion portion 11 than the rigidity changeable structure 172 in the rigidity changeable range of the insertion portion 11. The rigidity changeable structure 171 is connected with a rigidity control unit 362 through a pulling member TMA such as a wire. The rigidity changeable structure 171 is provided near a source coil SD (not illustrated) corresponding to one of the plurality of source coils included in the source coil group 113. Specifically, the rigidity changeable structure 171 can change the rigidity of a segment PD (not illustrated) separated as a section corresponding to the position of the source coil SD in the rigidity changeable range of the insertion portion 11.

The rigidity changeable structure 172 is disposed being positioned to the central part of the rigidity changeable range of the insertion portion 11, in other words, a central part of the rigidity changeable mechanism 162. The rigidity changeable structure 172 is connected with the rigidity control unit 362 through a pulling member TMB such as a wire. The rigidity changeable structure 172 is provided near a source coil SE (not illustrated) corresponding to one source coil disposed at a position closer to the proximal end side of the insertion portion 11 than the source coil SD among the plurality of source coils included in the source coil group 113. Specifically, the rigidity changeable structure 172 can change the rigidity of a segment PE (not illustrated) separated as a section corresponding to the position of the source coil SE in the rigidity changeable range of the insertion portion 11.

For example, the rigidity changeable structure 173 is disposed being positioned to a position closer to the proximal end portion of the insertion portion 11 than the rigidity changeable structure 172 in the rigidity changeable range of the insertion portion 11. The rigidity changeable structure 173 is connected with the rigidity control unit 362 through a pulling member TMC such as a wire. The rigidity changeable structure 173 is provided near a source coil SF (not illustrated) corresponding to one source coil disposed at a position closer to the proximal end side of the insertion portion 11 than the source coil SE among the plurality of source coils included in the source coil group 113. Specifically, the rigidity changeable structure 173 can change the rigidity of a segment PF (not illustrated) separated as a section corresponding to the position of the source coil SF in the rigidity changeable range of the insertion portion 11.

With the configuration of the rigidity changeable mechanism 162 as described above, the entire rigidity changeable range of the insertion portion 11 is divided into three segments, namely, the segment PD corresponding to the position of the source coil SD, the segment PE corresponding to the position of the source coil SE, and the segment PF corresponding to the position of the source coil SF. With the configuration of the rigidity changeable mechanism 162 as described above, the rigidity changeable structure 171 is disposed at a position corresponding to the segment PD, the rigidity changeable structure 172 is disposed at a position corresponding to the segment PE, and the rigidity changeable structure 173 is disposed at a position corresponding to the segment PF. With the configuration as described above, the rigidity changeable mechanism 162 can change rigidity for each of the three segments PD, PE, and PF.

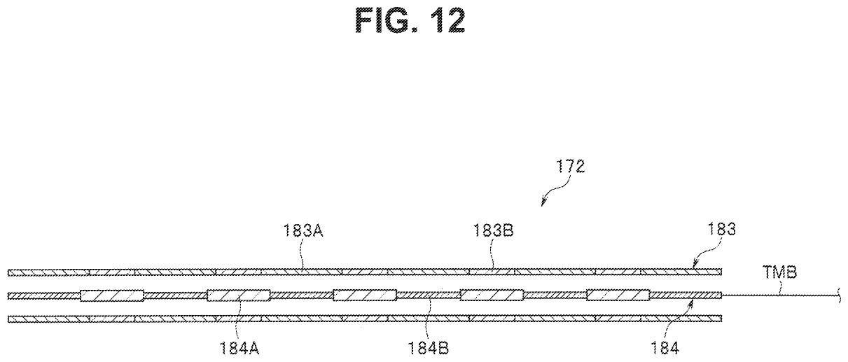

As illustrated in FIG. 12, the rigidity changeable structure 172 includes a sheath member 183 and a bar member 184. FIG. 12 is a diagram for description of a configuration of a rigidity changeable mechanism according to the modification of the second embodiment.

The sheath member 183 has, for example, an elongated cylindrical shape. The sheath member 183 is disposed being fixed inside the insertion portion 11 (flexible tube portion 11C). A rigid portion 183A (rigid site) that is relatively hard and a flexible portion 183B (flexible site) that is relatively soft are alternately provided in the sheath member 183.

The bar member 184 is disposed being inserted into an internal space of the sheath member 183. The bar member 184 has, for example, an elongated cylinder shape. The bar member 184 is disposed being slidable inside the sheath member 183. An end part of the bar member 184 on the proximal end side is connected with the rigidity control unit 362 through the pulling member TMB. The bar member 184 together with the pulling member TMB can move forward and backward inside the sheath member 183. In addition, a large diameter portion 184A (large diameter site) having a relatively large diameter and a small diameter portion 184B (small diameter site) having a relatively small diameter are alternately provided in a longitudinal direction of the bar member 184.

The rigidity control unit 362 is provided to the main body device 30A in place of the rigidity control unit 342. The rigidity control unit 362 is configured to perform operation for controlling the rigidity changeable mechanism 162 based on the insertion shape information outputted from the insertion shape information acquisition unit 402. As illustrated in FIG. 11, the rigidity control unit 362 includes motors 371A, 371B, and 371C, encoders 372A, 372B, and 372C, a memory 373, and a control circuit 374.

The motor 371A is connected with the rigidity changeable structure 171 (the end part of the bar member 184 on the proximal end side in the rigidity changeable structure 171) through the pulling member TMA. The motor 371A can change a pulling amount of the pulling member TMA (length by which the pulling member TMA is wound) by rotating in accordance with control by the control circuit 374.

The motor 371B is connected with the rigidity changeable structure 172 (the end part of the bar member 184 on the proximal end side in the rigidity changeable structure 172) through the pulling member TMB. The motor 371B can change a pulling amount of the pulling member TMB (length by which the pulling member TMB is wound) by rotating in accordance with control by the control circuit 374.

The motor 371C is connected with the rigidity changeable structure 173 (the end part of the bar member 184 on the proximal end side in the rigidity changeable structure 173) through the pulling member TMC. The motor 371C can change a pulling amount of the pulling member TMC (length by which the pulling member TMC is wound) by rotating in accordance with control by the control circuit 374.

For example, the encoder 372A is configured to detect a current rotational amount and a current rotational direction of the motor 371A as a rotation state of the motor 371A and output, to the control circuit 374, rotation state information indicating the detected rotation state of the motor 371A.

For example, the encoder 372B is configured to detect a current rotational amount and a current rotational direction of the motor 371B as a rotation state of the motor 371B and output, to the control circuit 374, rotation state information indicating the detected rotation state of the motor 371B.

For example, the encoder 372C is configured to detect a current rotational amount and a current rotational direction of the motor 371C as a rotation state of the motor 371C and output, to the control circuit 374, rotation state information indicating the detected rotation state of the motor 371C.

The memory 373 stores rigidity control information used for control of the motors 371A, 371B, and 371C by the control circuit 374. Specifically, the memory 373 stores the rigidity control information including, for example, information indicating the rigidity changeable range of the insertion portion 11, information that can specify the three segments PD, PE, and PF included in the rigidity changeable range, and information indicating a threshold value corresponding to a predetermined parameter calculated for control of the rigidity changeable mechanism 162.

The control circuit 374 is configured to control the rotational amount and rotational direction of each of the motors 371A, 371B, and 371C based on the rotation state information outputted from the encoders 372A, 372B, and 372C, the rigidity control information read from the memory 373, and the insertion shape information outputted from the insertion shape information acquisition unit 402.

Specifically, a flexible tube insertion device according to the present modification includes the insertion portion 11, the rigidity changeable mechanism 162, and the rigidity control unit 362.

With the above-described configuration, the rigidity changeable mechanism 162 can perform, in accordance with control by the rigidity control unit 362, operation for sequentially increasing the bending rigidity of the rigidity changeable range of the insertion portion 11 in the direction from the central part of the rigidity changeable range to each end part of the rigidity changeable range.

With the above-described configuration, the rigidity changeable mechanism 162 can change bending rigidity for each of a plurality of segments into which the entire rigidity changeable range of the insertion portion 11 is divided.

With the above-described configuration, the bar member 184 of the rigidity changeable mechanism 162 can change the position of the bar member 184 relative to the sheath member 183 in accordance with control by the rigidity control unit 362.

With the above-described configuration, the rigidity control unit 362 can perform, on the rigidity changeable mechanism 162, control for changing the insertion shape of the insertion portion 11 being inserted into a subject based on the rigidity control information read from the memory 373 and the insertion shape information outputted from the insertion shape information acquisition unit 402.

With the above-described configuration, for example, when control for rotating the motor 371B so that the pulling amount of the pulling member TMB becomes equal to a pulling amount PX is performed by the control circuit 374, the relative positional relation between the sheath member 183 and the bar member 184 is changed, and accordingly, the position of the rigid portion 183A and the position of the large diameter portion 184A are aligned with each other, and the position of the flexible portion 183B and the position of the small diameter portion 184B are aligned with each other. Thus, as the pulling amount of the pulling member TMB becomes equal to the pulling amount PX, the rigidity of the segment PE in the rigidity changeable range of the insertion portion 11 decreases.

With the above-described configuration, for example, when control for rotating the motor 371B so that the pulling amount of the pulling member TMB becomes equal to a pulling amount PZ (>PX) is performed by the control circuit 374, the relative positional relation between the sheath member 183 and the bar member 184 is changed, and accordingly, the position of the rigid portion 183A and the position of the small diameter portion 184B are aligned with each other, and the position of the flexible portion 183B and the position of the large diameter portion 184A are aligned with each other. Thus, as the pulling amount of the pulling member TMB become equal to the pulling amount PZ, the rigidity of the segment PE in the rigidity changeable range of the insertion portion 11 increases.

Subsequently, effects of the present modification will be described below.

The control circuit 374 specifies each of the segments PD, PE, and PF included in the rigidity changeable range of the insertion portion 11 based on the rigidity control information read from the memory 373 and the insertion shape information outputted from the insertion shape information acquisition unit 402. The control circuit 374 calculates a curvature CVE of the segment PE specified as described above and determines whether the calculated curvature CVE is equal to or larger than a threshold value TVE. In such a case, the rigidity control information includes the threshold value TVE corresponding to the curvature CVE of the segment PE positioned at the central part of the rigidity changeable range of the insertion portion 11.

For example, when having acquired a determination result that the curvature CVE is smaller than the threshold value TVE, the control circuit 374 performs, based on the rotation state information outputted from the encoders 372A, 372B, and 372C, control for rotating the motors 371A, 371B, and 371C so that the pulling amounts of the pulling members TMA, TMB, and TMC each become equal to the pulling amount PX. Through such control by the control circuit 374, the rigidity of the entire rigidity changeable range of the insertion portion 11 uniformly decreases.

For example, when having acquired a determination result that the curvature CVE is equal to or larger than the threshold value TVE, the control circuit 374 performs, based on the rotation state information outputted from the encoder 372B, control for rotating the motor 371B so that the pulling amount of the pulling member TMB becomes equal to the pulling amount PZ. After a certain time has elapsed since the control for rotating the motor 371B so that the pulling amount of the pulling member TMB becomes equal to the pulling amount PZ is performed, the control circuit 374 simultaneously performs, based on the rotation state information outputted from the encoders 372A and 372C, control for rotating the motor 371A so that the pulling amount of the pulling member TMA becomes equal to the pulling amount PZ, and control for rotating the motor 371C so that the pulling amount of the pulling member TMC becomes equal to the pulling amount PZ. Through such control by the control circuit 374, the rigidity of the segment PE in the rigidity changeable range of the insertion portion 11 relatively increases and the rigidity of the segments PD and PF in the rigidity changeable range relatively decreases in a duration until a certain time elapses since a timing TC at which the determination result that the curvature CVE is equal to or larger than the threshold value TVE is obtained. Through the above-described control by the control circuit 374, the rigidity of the entire rigidity changeable range of the insertion portion 11 uniformly increases in a duration following a timing TD after a certain time has elapsed since the timing TC.

Specifically, the control circuit 374 performs control that operates the rigidity changeable mechanism to increase the bending rigidity of the segment PE belonging to the central part of the rigidity changeable range of the insertion portion 11 and then sequentially increase the bending rigidity toward the segments PD and PF belonging to both end parts of the rigidity changeable range.

As described above, according to the present modification, force for deforming the insertion portion 11 closer to a straight line shape can be generated by changing the relative positional relation between the sheath member 183 and the bar member 184. In addition, according to the present modification, force for deforming the insertion portion 11 closer to a straight line shape can be substantially simultaneously generated in two directions, namely, a direction from the central part of the rigidity changeable mechanism 162 to the distal end portion of the insertion portion 11 and a direction from the central part of the rigidity changeable mechanism 162 to the proximal end portion of the insertion portion 11. Thus, according to the present modification, since rigidity change along with the above-described force generation substantially simultaneously occurs in the above-described two directions, it is possible to efficiently straighten the insertion portion 11 being buckled inside a subject without providing an excessive load to the subject, and it is possible to increase the rigidity of part of the rigidity changeable range of the insertion portion 11, which is positioned on the distal end side of a bending site inside the subject.

Specifically, according to the present modification, for example, when buckling as illustrated in FIG. 6A has occurred to the insertion portion 11 being inserted into the intestinal canal of the subject, it is possible to efficiently straighten the insertion portion 11. According to the present modification, for example, when the insertion portion 11 being inserted into the intestinal canal of the subject passes through a bending site as illustrated in FIG. 6B, it is possible to improve thrust performance of the insertion portion 11 by increasing the rigidity of the rigidity changeable mechanism 162 in a state in which substantially the entire rigidity changeable range of the insertion portion 11 is positioned on the distal end side of the bending site. In such a case, the shape of part of the insertion portion 11, which is positioned on the distal end side of the bending site deforms closer to a straight line shape along with increase of the rigidity of part of the rigidity changeable mechanism 132, which is positioned on the distal end side of the bending site inside the subject. Thus, according to the present modification, it is possible to improve insertion performance when the elongated insertion portion having flexibility is inserted into a deep part in the subject.

According to the present modification, for example, the rigidity control information may include a threshold value TRE corresponding to a curvature radius CRE of a segment SE positioned at the central part of the rigidity changeable range of the insertion portion 11. In such a case, control for rotating each motor so that the pulling amount of the corresponding pulling member becomes equal to the pulling amount PX may be performed when a determination result that the curvature radius CRE is equal to or smaller than the threshold value TRE is obtained, and control for rotating each motor so that the pulling amount of the corresponding pulling member becomes equal to the pulling amount PZ may be performed when a determination result that the curvature radius CRE is larger than the threshold value TRE is obtained.

According to the present modification, for example, when the rigidity changeable range of the insertion portion 11 is divided into four or more segments, control may be performed by considering that a plurality of segments belong to the central part of the rigidity changeable range.

Specifically, according to the present modification, the rigidity control unit 362 may perform control that operates the rigidity changeable mechanism 162 to increase the bending rigidity of one or more segments belonging to the central part of the rigidity changeable range of the insertion portion 11 among a plurality of segments included in the rigidity changeable range and then sequentially increase the bending rigidity toward segments belonging to both end parts of the rigidity changeable range among the plurality of segments. According to the present modification, when the curvature of a predetermined segment included in one or more segments belonging to the central part of the rigidity changeable range of the insertion portion 11 is equal to or larger than a threshold value or when the curvature radius of the predetermined segment is equal to or smaller than a threshold value, the rigidity control unit 362 may perform predetermined control for displacing the bar member 184 so that the position of the flexible portion 183B and the position of the large diameter portion 184A coincide with each other in one or more rigidity changeable structures disposed at the central part of the rigidity changeable range and then may perform the predetermined control on each rigidity changeable structure other than the one or more rigidity changeable structures in ascending order of distance to the central part of the rigidity changeable range.

The present invention is not limited to the above-described embodiments and may be modified and applied in various kinds of manners without departing from the scope of the invention.

What is claimed is:

1. A flexible tube insertion device comprising:
an insertion portion having flexibility and an elongated shape;
a rigidity changeable mechanism extending in a longitudinal direction of the insertion portion in a rigidity changeable range corresponding to at least a partial range of the insertion portion, the rigidity changeable mechanism being configured to be capable of changing bending rigidity of the rigidity changeable range; and
a processor configured to be capable of performing, on the rigidity changeable mechanism, control for changing an insertion shape of the insertion portion being inserted into a subject, wherein
the rigidity changeable mechanism is configured to perform, in accordance with control by the processor, operation for sequentially increasing bending rigidity of the rigidity changeable range in a direction from a central part of the rigidity changeable range to each end part of the rigidity changeable range.

2. The flexible tube insertion device according to claim 1, wherein the rigidity changeable mechanism is configured to change bending rigidity of a whole of the rigidity changeable range as one segment and perform, in accordance with control by the processor, operation that increases bending rigidity at a central part of the one segment and then sequentially increases bending rigidity toward each end part of the one segment.

3. The flexible tube insertion device according to claim 2, wherein the rigidity changeable mechanism includes:
a coil heater having such a coil shape that wire winding density gradually decreases in a direction from the central part of the one segment to the each end part, the coil heater being configured to generate heat in accordance with control by the processor; and
a shape-memory member disposed being inserted into an internal space of the coil heater and configured to be capable of changing elasticity in accordance with the heat generated from the coil heater.

4. The flexible tube insertion device according to claim 3, further comprising an insertion shape detection device configured to acquire insertion shape information indicating the insertion shape of the insertion portion being inserted into the subject, wherein
the processor calculates a curvature or curvature radius of the one segment based on the insertion shape information obtained by the insertion shape detection device, and when the calculated curvature is equal to or larger than a threshold value or when the calculated curvature radius is equal to or smaller than a threshold value, the processor performs control for generating heat from the coil heater.

5. The flexible tube insertion device according to claim 2, wherein the rigidity changeable mechanism includes:
a sheath member that is disposed being fixed inside the insertion portion and has an outer cover, a plurality of slits having lengths that gradually increase in a direction from the central part of the one segment to the each end part being formed on the outer cover; and
a bar member that is disposed being slidable inside the sheath member and has a large diameter site and a small diameter site, the large diameter site having a length equal to or larger than a maximum length among the lengths of the plurality of slits and having a relatively large diameter, the small diameter site having a relatively small diameter, the large diameter site and the small diameter site being alternately provided, a position of the bar member relative to the sheath member being changed in accordance with control by the processor.

6. The flexible tube insertion device according to claim 5, further comprising an insertion shape detection device configured to acquire insertion shape information indicating the insertion shape of the insertion portion being inserted into the subject, wherein
the processor calculates a curvature or curvature radius of the one segment based on the insertion shape information obtained by the insertion shape detection device, and when the calculated curvature is equal to or larger than a threshold value or when the calculated curvature radius is equal to or smaller than a threshold value, the processor performs control for displacing the bar member so that positions of the plurality of slits coincide with a position of the large diameter site.

7. The flexible tube insertion device according to claim 1, wherein
the rigidity changeable mechanism is configured to change bending rigidity for each of a plurality of segments into which a whole of the rigidity changeable range is divided, and
the processor performs control that operates the rigidity changeable mechanism to increase bending rigidity of one or more segments belonging to the central part of the rigidity changeable range among the plurality of segments and then sequentially increase bending rigidity toward segments belonging to both end parts of the rigidity changeable range among the plurality of segments.

8. The flexible tube insertion device according to claim 7, wherein the rigidity changeable mechanism includes:
a plurality of coil heaters provided at positions corresponding to the plurality of respective segments and configured to generate heat in accordance with control by the processor; and
a shape-memory member disposed being inserted into internal spaces of the plurality of coil heaters and configured to be capable of changing elasticity in accordance with heat generated from at least one of the plurality of coil heaters.

9. The flexible tube insertion device according to claim 8, further comprising an insertion shape detection device configured to acquire insertion shape information indicating the insertion shape of the insertion portion being inserted into the subject, wherein
the processor calculates a curvature or curvature radius of a predetermined segment included in the one or more segments based on the insertion shape information obtained by the insertion shape detection device, and when the calculated curvature is equal to or larger than a threshold value or when the calculated curvature radius is equal to or smaller than a threshold value, the processor performs predetermined control for generating heat from one or more coil heaters disposed at the central part of the rigidity changeable range and then performs the predetermined control on each of the coil heaters other than the one or more coil heaters in ascending order of distance to the central part of the rigidity changeable range.

10. The flexible tube insertion device according to claim 7, wherein
the rigidity changeable mechanism includes a plurality of rigidity changeable structures provided at positions corresponding to the plurality of respective segments, and
each of the plurality of rigidity changeable structures includes:
a sheath member that is disposed being fixed inside the insertion portion and has a rigid site that is relatively hard and a flexible site that is relatively soft, the rigid site and the flexible site being alternately provided; and
a bar member that is disposed being slidable inside the sheath member and has a large diameter site and a small diameter site, the large diameter site having a relatively large diameter, the small diameter site having a relatively small diameter, the large diameter site and the small diameter site being alternately provided, a position of the bar member relative to the sheath member being changed in accordance with control by the processor.

11. The flexible tube insertion device according to claim 10, further comprising an insertion shape detection device configured to acquire insertion shape information indicating the insertion shape of the insertion portion being inserted into the subject, wherein
the processor calculates a curvature or curvature radius of a predetermined segment included in the one or more segments based on the insertion shape information obtained by the insertion shape detection device, and when the calculated curvature is equal to or larger than a threshold value or when the calculated curvature radius is equal to or smaller than a threshold value, the processor performs predetermined control for displacing the bar member in each of one or more rigidity changeable structures disposed at the central part of the rigidity changeable range so that a position of the flexible site and a position of the large diameter site coincide with each other, and then performs the predetermined control on each of the rigidity changeable structures other than the one or more rigidity changeable structures in ascending order of distance to the central part of the rigidity changeable range.

12. An endoscope system comprising:
an endoscope including
an insertion portion having flexibility and an elongated shape, and
a rigidity changeable mechanism extending in a longitudinal direction of the insertion portion in a rigidity changeable range corresponding to at least a partial range of the insertion portion, the rigidity changeable mechanism being configured to be capable of changing bending rigidity of the rigidity changeable range; and
a processor configured to be capable of performing, on the rigidity changeable mechanism, control for changing an insertion shape of the insertion portion being inserted into a subject, wherein
the rigidity changeable mechanism is configured to perform, in accordance with control by the processor, operation for sequentially increasing bending rigidity of the rigidity changeable range in a direction from a central part of the rigidity changeable range to each end part of the rigidity changeable range.

13. A flexible tube insertion method of inserting an insertion portion having flexibility and an elongated shape into a subject, the flexible tube insertion method comprising:
inserting the insertion portion into the subject; and
changing, by a rigidity changeable mechanism provided to the insertion portion, an insertion shape of the insertion portion being inserted into the subject by sequentially increasing bending rigidity of a rigidity changeable range corresponding to at least a partial range of the insertion portion in a direction from a central part of the rigidity changeable range to each end part of the rigidity changeable range.
